(19) United States
(12) Patent Application Publication
(10) Pub. No.: US 2017/0152200 A1
(43) Pub. Date: Jun. 1, 2017

(54) Zeolite Catalyst for Lignin to Phenol Conversion

(71) Applicant: Sabic Global Technologies, B.V., BERGEN OP ZOOM (NL)

(72) Inventors: Zhiqiang MA, Zurich (CH); Ashim Kumar GHOSH, Houston, TX (US); Jeroen VAN BOKHOVEN, Zurich (CH)

(21) Appl. No.: 15/333,961

(22) Filed: Oct. 25, 2016

Related U.S. Application Data
(60) Provisional application No. 62/260,956, filed on Nov. 30, 2015.

(51) Int. Cl. C07C 37/54; B01J 35/02; B01J 35/10; B01J 29/00

(52) U.S. Cl. CPC C07C 37/54; B01J 29/00; B01J 35/023; B01J 35/1057

(57) ABSTRACT

A method for converting lignin to a phenol product, the method comprising contacting a zeolite catalyst with a lignin under reaction conditions sufficient to produce the phenol product at a yield of equal to or greater than about 50%. A method for converting lignin to a mixed phenol product, the method comprising contacting a large-pore zeolite catalyst with a Kraft lignin under reaction conditions comprising a reaction temperature of from about 550° C. to about 850° C. to produce the mixed phenol product at a yield of equal to or greater than about 50%.

### CROSS-REFERENCE TO RELATED APPLICATIONS

The present application is a non-provisional of and claims priority to U.S. Provisional Patent Application No. 62/260,956 filed Nov. 30, 2015 and entitled “Zeolite Catalyst for Lignin to Phenol Conversion,” which application is incorporated by reference herein in its entirety.

### TECHNICAL FIELD

This disclosure relates to zeolite catalysts. More particularly the present disclosure relates to the use of zeolite catalysts for lignin to phenol conversion.

### BACKGROUND

The depletion of fossil fuels has spurred interest in the development of alternative feedstocks for the production of molecules utilized as building blocks in a variety of chemical applications. Biomass, such as lignin, is one possible renewable alternative feedstock for such materials. However, attempts to use lignin as a feedstock for the production of chemicals (e.g., aromatics) continue to prove commercially impractical due to the high costs of reagents and equipment, as well as the limited conversion to the desired end products.

Thus, there is an ongoing need for the development of new materials and processes for the conversion of lignin to higher value chemicals.

### SUMMARY

Disclosed herein is a method for converting lignin to a phenol product, the method comprising contacting a zeolite catalyst with a lignin under reaction conditions sufficient to produce the phenol product at a yield of equal to or greater than about 50%.

Also disclosed herein is a method for converting lignin to a mixed phenol product, the method comprising contacting a large-pore zeolite catalyst with a Kraft lignin under reaction conditions comprising a reaction temperature of from about 550° C. to about 850° C. to produce the mixed phenol product at a yield of equal to or greater than about 50%.

### DESCRIPTION OF THE DRAWINGS

FIG. 1 is a schematic embodiment of a system of the type disclosed herein.

FIGS. 2a-2c are graphs depicting the effect of the structure of zeolites with similar Si/Al ratio on the yield of products and product distribution.

FIGS. 3a-3c are graphs depicting the effect of Si/Al in the zeolite catalyst on the yield of products and product and distribution.

FIGS. 4a-4c are graphs depicting the effect of the lignin to catalyst ratio on the yield of products and product distribution.

FIGS. 5a-5c are graphs depicting the effect of pretreatment of the lignin on the yield of products and product distribution.

FIGS. 6a-6c are graphs depicting the effect of temperature on the yield of products and product distribution.

FIGS. 7a-7c are graphs depicting the effect of mixed FAU (15) and BEA (33) zeolites on the yield of products and product distribution.

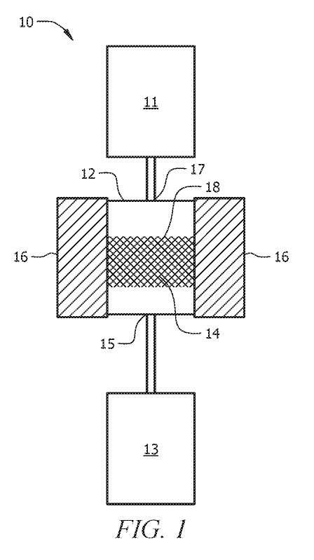

### DETAILED DESCRIPTION

Disclosed herein are methods and compositions for the conversion of biomass to higher value chemical products. In an embodiment, the biomass comprises lignin and the higher value chemical product comprises phenols. Methods of the present disclosure may comprise contacting lignin with a zeolite catalyst under conditions suitable for the conversion of lignin to one or more aromatic products such as phenol. In some embodiments, the present disclosure results in the formation of a mixture of phenols herein referred to as a “mixed phenol product.”

In an embodiment, the biomass comprises lignin. Lignin, shown in Structure I, is a phenyl-propanoid (C9) polyphenol mainly linked by arylglycerol ether bonds between the nonomeric phenolic p-coumaryl, coniferyl, and sinapyl alcohol units. The structure of lignin is characteristically a random three-dimensional network polymer exhibiting non-repetitive units and no repetitive bonding patterns.

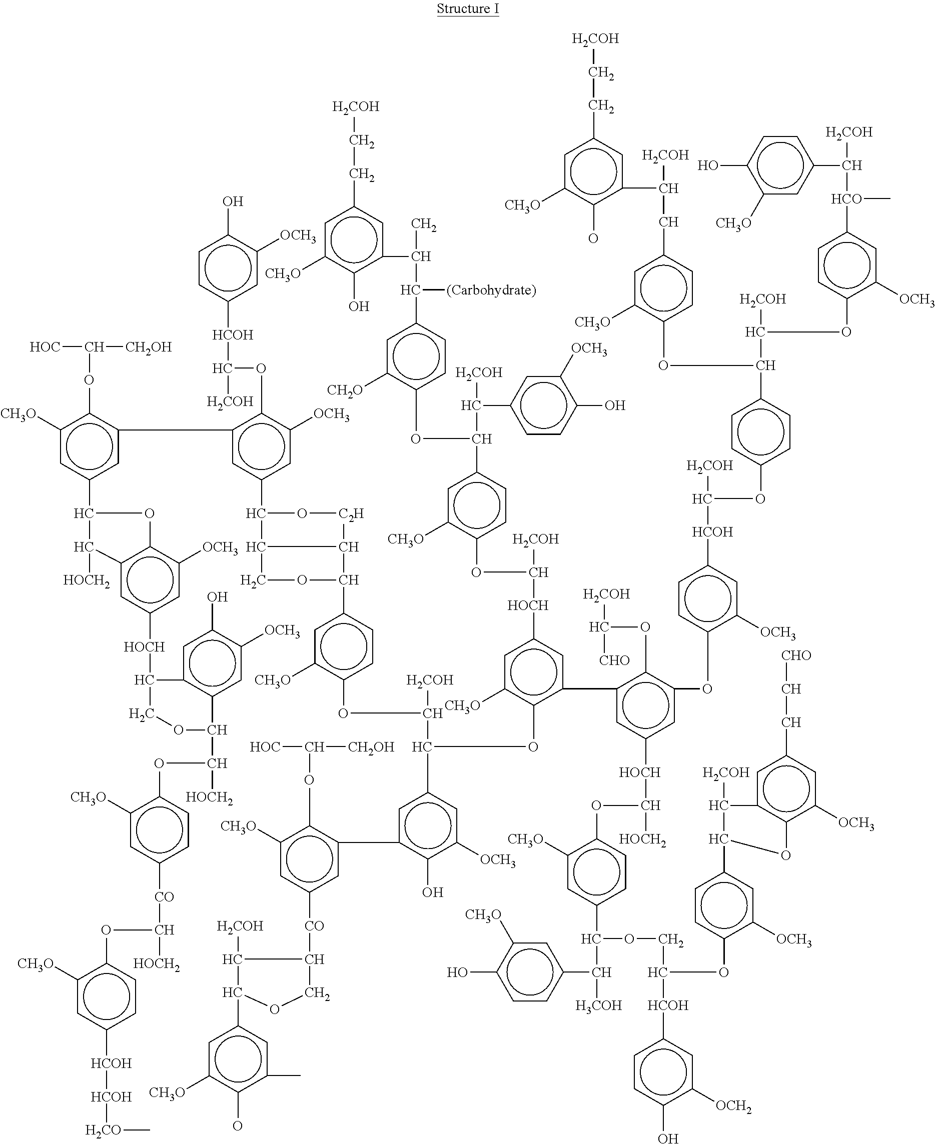

Lignin constitutes up to 30% of the weight and 40% of the energy content of lignocellulosic biomass (e.g., wood) with the remainder of the biomass being cellulose and hemicellulose. Lignin suitable for use in the present disclosure can be obtained from the lignocellulosic biomass using any suitable methodology. For example, lignin suitable for use the present disclosure may comprise Kraft lignin.

Kraft lignin refers to lignin obtained via the Kraft pulping process which involves treatment of the lignocellulosic biomass (e.g., wood) with sulfur-containing compounds (e.g., sulfide, sulfhydryl, and polysulfide) at high pH and at temperatures ranging from 150° C. to 180° C. The solubilized lignin is localized in the spent pulping liquor (“black liquor”) along with most of the hemicellulose and subsequently recovered by precipitation. Kraft lignin is characterized as being soluble in alkali and strongly polar organic solvents (e.g., dimethylsulfoxide, dimethylformamide). In an embodiment the lignin is Kraft lignin having a number average molecular weight ranging from about 1000 g/mol to about 3000 g/mol and a polydispersity index ranging from about 2 to about 4. The number average molecular weight (Mₙ) is defined by Equation 1

Mₙ=ΣᵢNᵢMᵢ/ΣᵢNᵢ

wherein Nᵢ is the number of polymer chains and Mᵢ is the molecular weight of a chain. The weight average molecular weight (M_{w}) is defined by Equation 2 where

M_{w}=ΣᵢNᵢMᵢ²/ΣᵢNᵢ

and the polydispersity index is defined by Equation 3 where

PDI=M_{w}/Mₙ

In an embodiment, a lignin suitable for use in the present disclosure comprises Organosolv lignin. Organosolv pulping to obtain lignin refers generally to the separation of lignocellulosic biomass through treatment with organic solvents. For example, Organosolv pulping to obtain lignin may involve the Allcel process which utilizes ethanol or ethanol-water as a solvent. Organosolv lignin can be obtain from the lignocellulosic biomass after contact with an appropriate solvent by solvent removal and recovery, or by precipitation of the lignin with water. Organosolv lignin may be characterized by an insolubility in water between pH 2 and pH 7 with the ability to dissolve in water at an alkali pH or in a polar organic solvent. Organosolv lignin is further characterized by a Mₙ of less than about 1000 g/mol and PDI ranging from about 2.4 to about 6.4.

In an embodiment, a lignin suitable for use in this disclosure comprises steam explosion lignin (SEL). SEL may be obtained by a process involving impregnation of the lignocellulosic biomass with steam under high pressure followed by a rapid pressure release. The lignin can be recovered by washing with an alkali solvent or extraction with an organic solvent. SEL may be characterized by the Mₙ ranging from about 600 g/mol to about 1000 g/mol and a PDI ranging from about 1.0 to about 3.0.

In an embodiment, a lignin suitable for use in the present disclosure is lignosulfonate lignin. Lignosulfonate lignin refers to lignin obtained via sulfite pulping that is typically carried out in a pH range of from about 2 to about 12 using divalent counterions such as calcium and magnesium in the lower pH range and sodium or ammonium coutnerions at higher pH ranges. Lignosulfonate lignin contains sulfur in the form of sulfonate groups present in the aliphatic side chains.

In an embodiment a lignin suitable for use in the present disclosure is selected from the group consisting of Kraft lignin, Organosolv lignin, lignosulfonate lignin, and SEL.

In an embodiment, a catalyst suitable for use in the present disclosure comprises a zeolite. Zeolites are crystalline solids made up of aluminum-substituted SiO₄ tetrahedral units joined together to form different ring and cage structures into a crystalline framework. The physical structure of a zeolite is characterized by both the extent of porosity and the presence of a large internal and external surface area. Further, in the crystalline structure of the zeolite there are pores and channels that may be interconnected. The dimensions and configuration of these pores and channels allow access of molecules of certain sizes or configurations, thus promoting the selectivity to certain products when a zeolite is used as the catalyst.

Zeolites suitable for use in the present disclosure may be naturally occurring or synthetic. In an embodiment, a zeolite suitable for use in the present disclosure contains medium or large pore sizes, having one, two, or three-dimensional pore structure. Herein “medium pore” zeolites refers to a zeolite having an average pore size that is in the range of from about 5 Å to about 7 Å. Herein “large pore” zeolite refers to a zeolite having an average pore size that is in the range of from about 7 Å to about 10 Å. It is possible that these ranges could overlap and a particular zeolite might be considered either a medium pore zeolite or a large pore zeolite. These ranges are in contrast to small pore zeolites which herein refer to zeolites having an average pore size of less than about 5 Å. Nonlimiting examples of medium and large pore zeolites suitable for use in the present disclosure include faujasite, ZSM-5, ZSM-11, ZSM-23, Silicalite, Mordenite, zeolite Beta, Zeolite-L, Zeolite X and Y, and combinations thereof. In an embodiment the zeolite comprises zeolite Beta, faujasite, or combinations thereof.

Zeolite Beta consists of an intergrowth of two distinct structures termed Polymorph A and Polymorph B. The polymorphs grow as two-dimensional sheets and the sheets randomly alternate between the two. Both polymorphs have a three dimensional network of 12-ring pores. The intergrowth of the polymorphs does not significantly affect the pores in two of the dimensions, but in the direction of the faulting, the pore becomes tortuous, but not blocked. Ball and stick representations of the framework structure of Polymorphs A and B are shown below.

In an embodiment, the zeolite catalyst comprises faujasite. Faujasite corresponds to the most open framework of all natural zeolites. About half of the unit-cell space is void in the dehydrated form. The structure consists of sodalite cages connected in a cubic manner over six-membered double rings (shown as Structure II).

Thus wide intersecting channels are formed parallel to <111> with an aperature of 7.4 Å. Approximately 50% of the cations reside in the sodalite cage bonded to three framework oxygens of the six-membered rings and additional H₂O molecules. The remaining cations and H₂O molecules are disordered in the large cavities.

In an embodiment the zeolite catalyst may be characterized by Formula I

M_{y/n}[SiₓAl_{y}O_{2(x+y)}] (I)

where M is a cation, n refers to the charge of the cation, and y/n is the number of cations. Silica and alumina atoms in the framework structure are referred to as T atoms. M can be a mono or divalent cation such as an alkali metal cation; alkaline earth metal cation, or combinations thereof and n is 1 or 2. The zeolite framework may contain gallium (Ga), boron (B), iron (Fe), indium (In), or combinations thereof as substitutions for at least some of the T atoms. In the embodiment, a zeolite catalyst suitable for use in the present disclosure has an x/y ratio ranging from about 5 to about 80, alternatively from about 7 to about 50, or alternatively from about 10 to about 30. If a T atom is substituted with another atom, for example Ga, then y in the x/y ratio is inclusive of the substituted atom, that is Si/(Al+Ga).

In an aspect the zeolite catalyst can have an average particle size of from about 0.05 μm to about 2 μm, alternatively from about 0.2 μm to about 0.7 μm, or alternatively from about 0.3 μm to about 0.5 μm. In an embodiment, a zeolite suitable for use in the present disclosure has an acid site density of from about 0.2 mmol/g of catalyst to about 2.8 mmol/g of catalyst, alternatively from about 0.3 mmol/g to about 2.1 mmol/g of catalyst, or alternatively from about 0.5 mmol/g to about 1.5 mmol/g of catalyst. Herein the acid site density refers to total acid site per gram of zeolite based on the Si/Al ratio.

In an embodiment a method of the present disclosure comprise treatment of lignin with an acidic material to form an acidified lignin. For example, lignin (e.g., Kraft lignin) may be contacted with an acidic material in an amount of from about 0.01 to about 0.22 g HCl/g Kraft lignin. In an embodiment, the acidic material is hydrochloric acid although other acids such as sulfonic or phosphoric may be employed. In an embodiment, the lignin is contacted with the acidic material (e.g., HCl) at a temperature in the range of from about 20° C. to about 70° C., for a time period of from about 2 hours to about 12 hours. The acidified lignin may be recovered by any suitable methodology such as by evaporation.

In an embodiment, the acidified lignin is subjected to pyrolysis in the presence of a zeolite catalyst of the type disclosed herein. Pyrolysis refers to the thermochemical decomposition of a substance (i.e., acidified lignin) in the absence of oxygen or halogens. Scheme I depicts an embodiment of the reaction where the reaction of the acidified lignin with the zeolite catalyst results in the production of a variety of mixed phenols of the type shown and other aromatics such as benzene, toluene, xylene and ethylbenzene (BTX-EB).

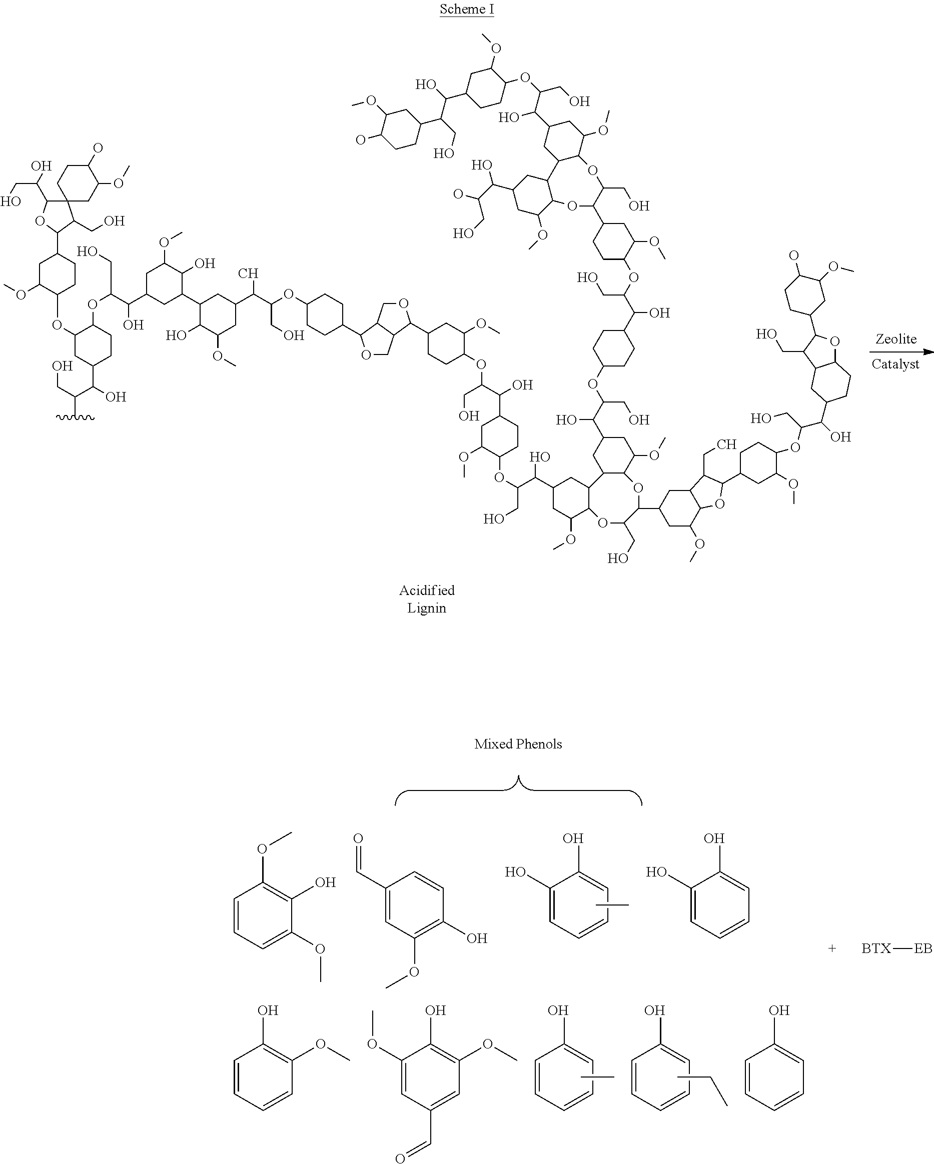

Conditions that may impact the extent of mixed phenol production from acidified lignin (as exemplified in Scheme I) include temperature, time, lignin concentration, type of zeolite, acid density (Si/Al ratio), and temperature ramp. The acidified lignin and zeolite catalyst may be contacted in a reaction mixture in amounts resulting in a lignin to catalyst weight ratio of about 1:1, alternatively about 2:1, or alternatively 1:2 at a temperature of from about 550° C. to about 850° C. for a time period ranging from about 5 seconds to about 60 seconds. The conversion of lignin to mixed phenols may be carried out at a pressure of about 14.7 psi, alternatively about 5.1 psi, or alternatively about 24.9 psi. The conditions for the conversion of lignin to mixed phenols may be varied based on the type or size of reactor. In some embodiments, the zeolite catalyst is regenerated after about 20 seconds of use in converting lignin to mixed phenols, and regeneration of the zeolite may be performed via calcination of the spent zeolites at temperature range of from about 500° C. to about 750° C. under air or oxygen atmosphere.

In an embodiment the methods disclosed herein result in a yield of mixed phenols of equal to or greater than about 50%, alternatively from about 5% to about 20%, or alternatively from about 30% to about 40%. Herein the yield is determined based on the amount of mixed phenol product/total amount of product multiplied by 100%. For example, the yield can be calculated based on the peak area of all mixed phenols/peak area of products (excluding water) detected in the gas chromatograph mass spectrometer (GCMS).

In an embodiment, the conversion of a feed comprising lignin (e.g., acidified Kraft lignin) using a crystalline catalyst (e.g., zeolite Beta) results in a product comprising mixed phenols as the major product.

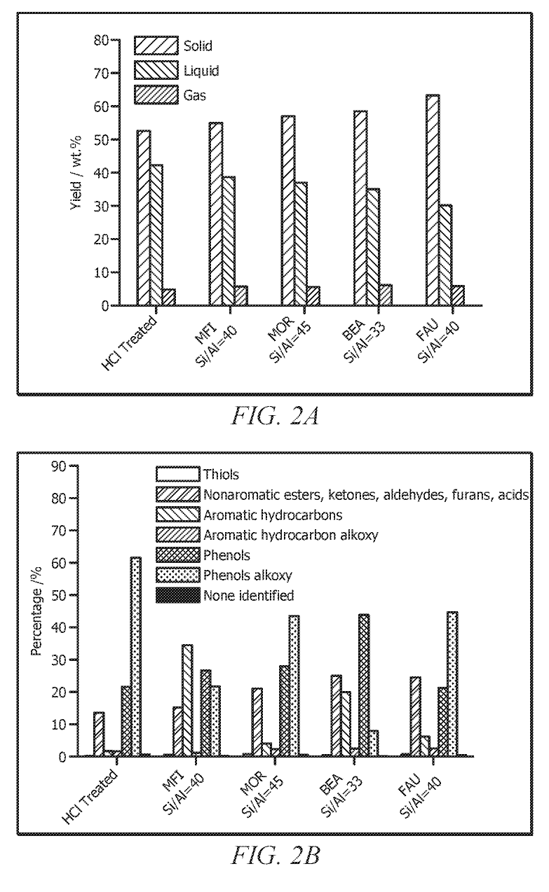

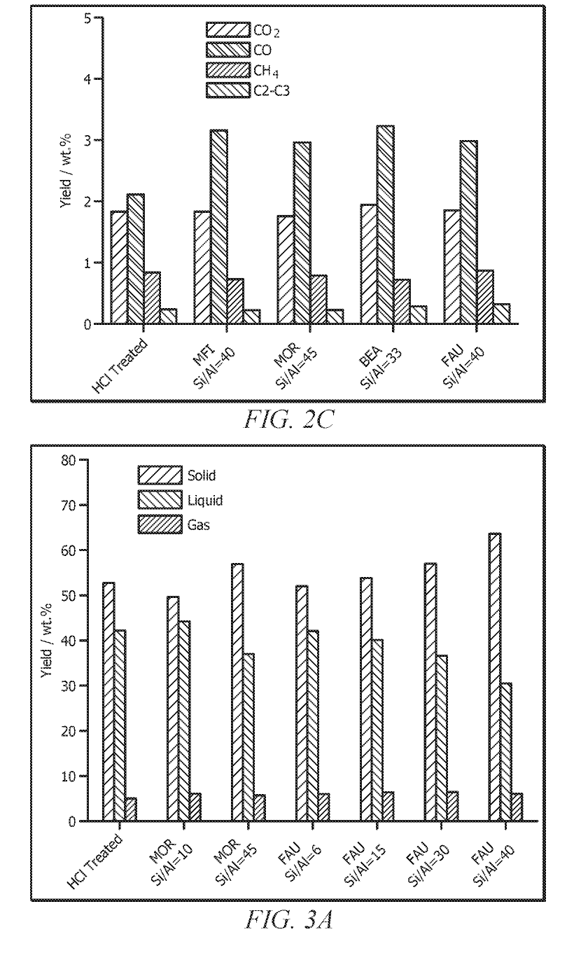

Referring to FIG. 2, a system 10 is illustrated, which can be used to convert lignin to mixed phenol products with the zeolite catalyst of the present disclosure. The system 10 can include a lignin source 11, a reactor 12, and a collection device 13. The lignin source 11 can be configured to be in fluid communication with the reactor 12 via an inlet 17 on the reactor, or pre-mixed with zeolite 14 with specific ratio in the reactor 12. As explained above, the lignin source can be configured such that it regulates the amount of lignin feed entering the reactor 12. The reactor 12 can include a reaction zone 18 having the catalyst (e.g., zeolite Beta or faujasite) 14 of the present disclosure. Non-limiting examples of reactors that can be used include fixed-bed reactors, fluidized bed reactors, bubbling bed reactors, slurry reactors, rotating kiln reactors, or any combinations thereof when two or more reactors are used. The amount of the zeolite catalyst 14 used can be modified as desired to achieve a given amount of product produced by the system 10. A non-limiting example of a reactor 12 that can be used is a fixed-bed reactor (e.g., a fixed-bed tubular stainless steel reactor which can be operated at atmospheric pressure). The reactor 12 can include an outlet 15 for products produced in the reaction zone 18. The collection device 13 can be in fluid communication with the reactor 12 via the outlet 15. Both the inlet 17 and the outlet 15 can be open and closed as desired. The collection device 13 can be configured to store, further process (e.g., separate and/or further treat/react), or transfer desired reaction products (e.g., mixed phenol product) for other uses. Still further, the system 10 can also include a heating source 16. The heating source 16 can be configured to heat the reaction zone 18 to a temperature sufficient (e.g., 550° C. to 850° C.) to convert the lignin in the lignin feed to mixed phenol products. A non-limiting example of a heating source 16 can be a temperature controlled furnace. Additionally, any unreacted lignin can be recycled and included in the lignin feed to further maximize the overall conversion of lignin to mixed phenol products. Further, certain products or byproducts such as BTX-EB can be separated and used in other processes to produce commercially valuable chemicals (e.g., aromatics). This increases the efficiency and commercial value of the lignin conversion process of the present disclosure. The methods of the present disclosure can further include collecting or storing the mixed phenol product along with using the mixed phenol product to produce a petrochemical or a polymer.

### EXAMPLES

The disclosure having been generally described, the following examples are given as particular embodiments of the disclosure and to demonstrate the practice and advantages thereof. It is understood that the examples are given by way of illustration and are not intended to limit the specification or the claims in any manner.

### Experimental Procedure

Lignin Pretreatments. 2 g of Kraft lignin dissolved in 10 mL H₂O, then 2 mL of 1 mol/L NaOH solution (6 mol/L HCl solution, or 30 wt. % of H₂O₂) was added under stirring for 6 h at room temperature. In case of two chemicals, each was taken for 1 mL.

Lignin Pyrolysis. The fast pyrolysis experiments were conducted using a platinum coil pyrolyzer (5150, CDS Analytical). The probe was a computer controlled resistively heated element (up to 20,000° C./s) which held an open ended quartz reactor. Before pyrolysis, lignin was ground into fine powder. In case of catalytic pyrolysis, lignin and the catalysts were physically mixed with lignin/catalyst weight ratio from 4:1 to 1:2. The powdered samples were accurately weighted and introduced into the reactor with loose quartz wool packing; during pyrolysis, vapors flowed from the open end of the quartz tube into a larger cavity, the pyrolysis interface, via a helium carrier gas stream before entering into the gas chromatograph.

Analytical. The carrier gas stream containing the pyrolysis products was directly injected into an Agilent 7890A gas chromatograph (GC) system through the pyrolysis interface and transfer line. The GC equipped with a thermal conductivity detector and an Agilent 5975C mass selective detector.

The oven was programmed to start at 40° C., hold for 5 min and then ramp at 10° C./min to a final temperature of 260° C. where it was held for 25 min. The injector temperature was 300° C. and a split ratio of 50:1 was used. The GC separation of pyrolysis vapors in the condensable gas range was done with a HP-5MS column (30 m×0.25 mm×0.25 μm) with helium as carrier gas (1.1 mL/min). Peak identification was done using the NISTO8 mass spectrum library. Pyrolysis vapors comprising condensable gases such as water and pyrolytic oil were classified as “liquid”. A total of over 50 compounds were detected by GC-MS (water not included). These products were further classified into five categories: aromatic hydrocarbons; aromatic hydrocarbon alkoxy; phenols; phenol alkoxy; thiols, nonaromatic esters, ketons, aldehydes, furans, acids; and none identified. The analysis of non-condensable gas range products was done simultaneously in the same equipment using Agilent Plot/Q and molecular sieve capillary columns. To quantify permanent gas yields, calibration curves were produced using a standard gas mixture comprising CO, CO₂, CH₄, and C₂-C₃ (containing C₂H₄, C₂H₆, C₃H₆, and C₃H₈) in Helium. These gaseous products were classified as “gas.” The residue formed in pyrolysis was classified as “solid.” The yield of solid was determined by a microbalance. In the residue, ash remained after pyrolysis in addition to coke/char.

### Example 1

The effect of utilizing zeolites with a differing structure but similar Si/Al ratio on the conversion of lignin to a mixed phenol product using the methodologies disclosed herein was investigated. The conditions for pyrolysis of lignin were 650° C. for 20 seconds using a temperature ramp of 20° C./ms and a lignin:catalyst ratio of 1:1. The lignin used was a Kraft lignin that had been pretreated with HCl. Specifically, all zeolite catalysts compared were in their H-form having Si/Al ratios as follows: MFI (Si/Al=40), mordenite (MOR) (Si/Al=45), zeolite Beta (BEA) (Si/Al=33) and faujasite (FAU) (Si/Al=40). FIG. 2a provides for the yield of solid, liquid, and gas components when using the different zeolites, or when using no zeolite catalyst (labeled HCl treated). FIG. 2b displays the liquid product distribution and FIG. 2c provides the gas product distribution.

### Example 2

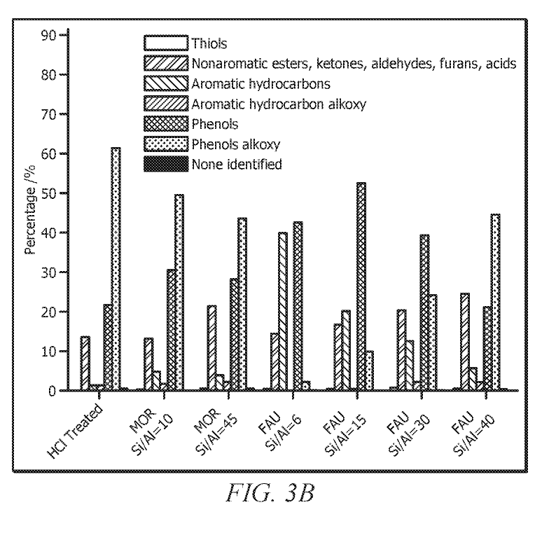

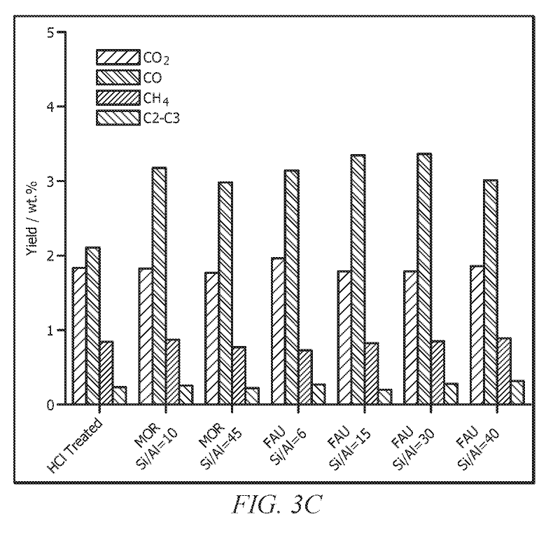

The effect of varying the Si/Al ratio of the zeolite catalyst on the conversion of lignin to a mixed phenol product was investigated. The conditions for pyrolysis of lignin were 650° C. for 20 seconds using a temperature ramp of 20° C./ms and a lignin:catalyst ratio of 1:1. Specifically for reactions employing mordenite as the catalyst the Si/Al ratio was varied from 10 to 45. For reactions employing faujasite the reactions were carried out with zeolite catalysts having a Si/Al ratio of 6, 15, 30, or 40. FIG. 3a provides for the yield of solid, liquid, and gas components when using the indicated zeolite catalyst at the indicated Si/Al ratio or when using no zeolite catalyst (labeled HCl treated). FIG. 3b displays the liquid product distribution and FIG. 3c provides the gas product distribution.

### Example 3

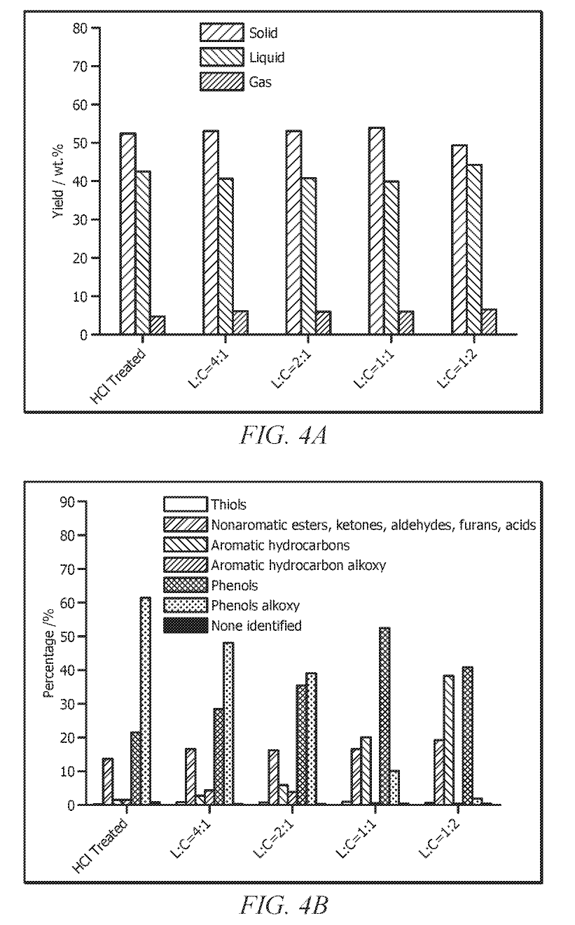

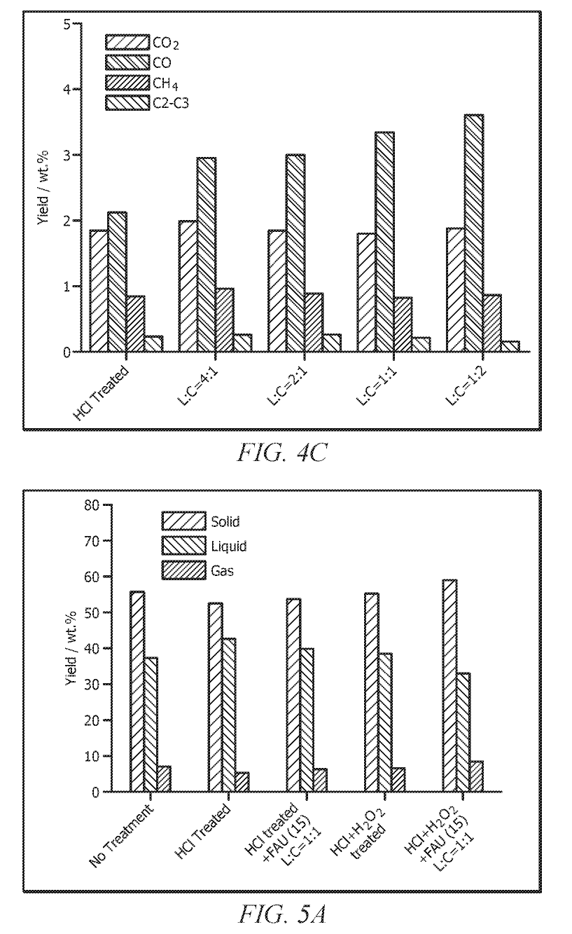

The effect of varying the lignin:catalyst ratio on the conversion of lignin to a mixed phenol product was investigated. The conditions for pyrolysis of lignin were 650° C. for 20 seconds using a temperature ramp of 20° C./ms. Specifically, the reactions used faujasite as the zeolite catalyst having a Si/Al ratio 15 and a lignin to catalyst ratio of 4:1, 2:1, 1:1, or 1:2, as indicated. FIG. 4a provides for the yield of solid, liquid, and gas components when at the different lignin:catalyst ratios, or when using no zeolite catalyst (labeled HCl treated). FIG. 4b displays the liquid product distribution and FIG. 4c provides the gas product distribution.

### Example 4

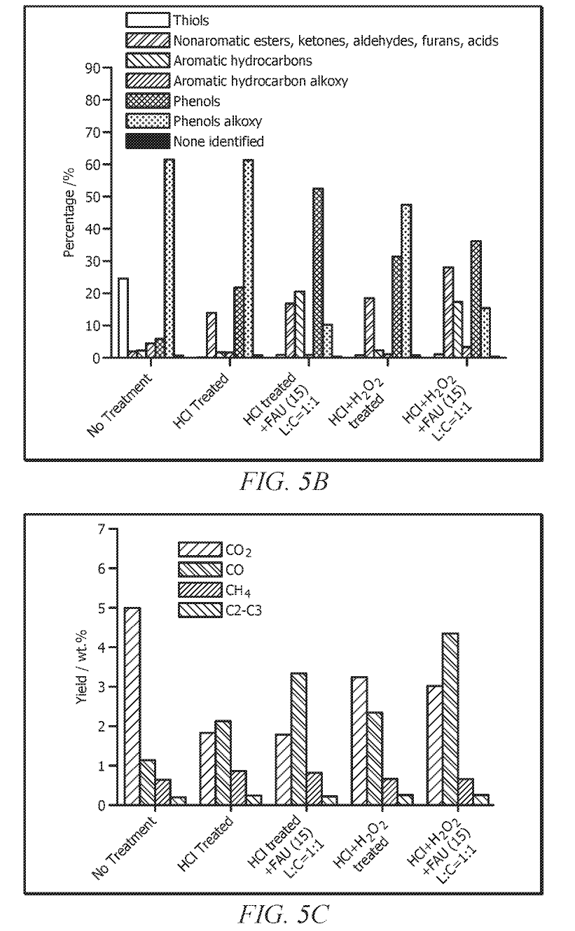

The effect of the type of lignin pretreatment on the conversion of lignin to a mixed phenol product was investigated. The conditions for pyrolysis of lignin were 650° C. for 20 seconds using a temperature ramp of 20° C./ms and the zeolite catalyst faujasite having a Si/Al ratio of 15 present in an amount to provide a lignin:catalyst ratio of 1:1. Specifically, reactions utilizing Kraft lignin that had not been pretreated were compared to reactions where the lignin had been (a) pretreated with HCl; (b) pretreated with HCl and contacted with faujasite having a Si/Al ratio of 15 at a lignin:catalyst ratio of 1:1; pretreated with HCl and H₂O₂; and pretreated with HCl and H₂O₂ and contacted with faujasite having a Si/Al ratio of 15 at a lignin:catalyst ratio of 1:1 FIG. 5a provides for the yield of solid, liquid, and gas components under the indicated conditions while FIG. 5b displays the liquid product distribution and FIG. 5c provides the gas product distribution.

### Example 5

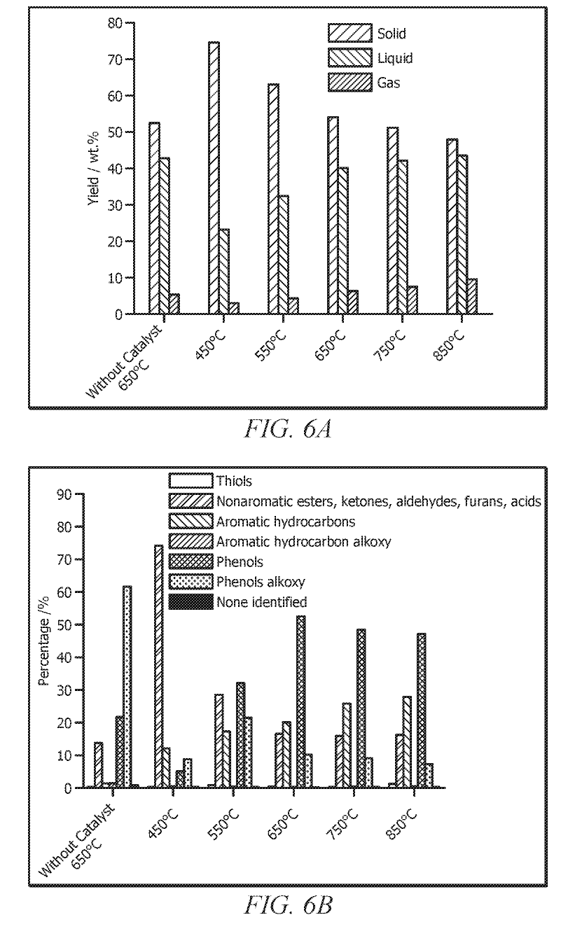

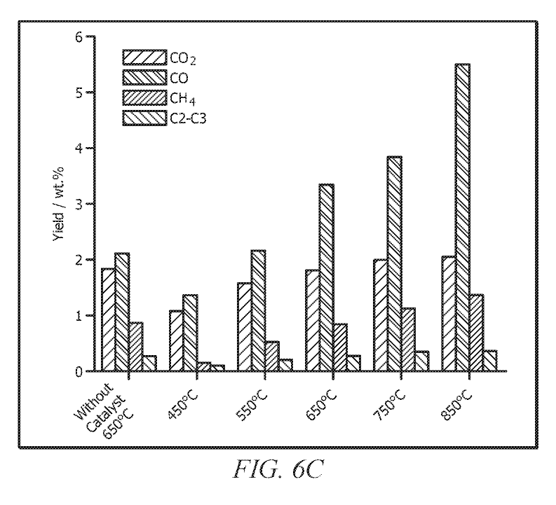

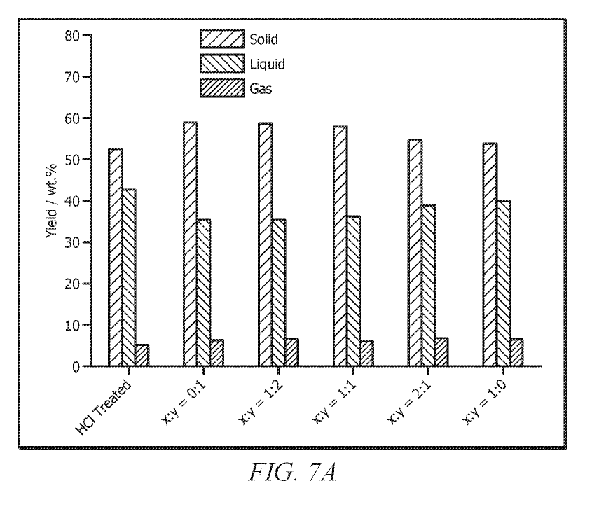

The effect of the pyrolysis temperature on the conversion of lignin to a mixed phenol product was investigated. The conditions for pyrolysis of lignin were the indicated temperature for 20 seconds using a temperature ramp of 20° C./ms using faujasite having a Si/Al ratio of 15 at a lignin:catalyst ratio of 1:1. Specifically, reactions were carried out at differing pyrolysis temperatures and compared to a reaction carried out at 650° C. in the absence of a zeolite catalyst. FIG. 6a provides for the yield of solid, liquid, and gas components at 450° C., 550° C., 650° C., 750° C., and 850° C. while FIG. 6b displays the liquid product distribution and FIG. 6c provides the gas product distribution.

### Example 6

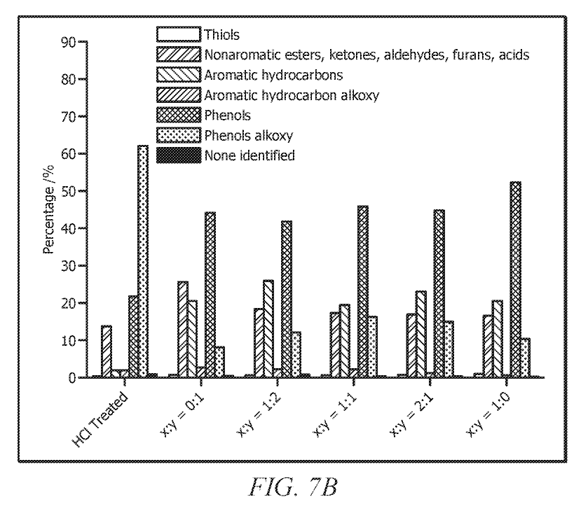

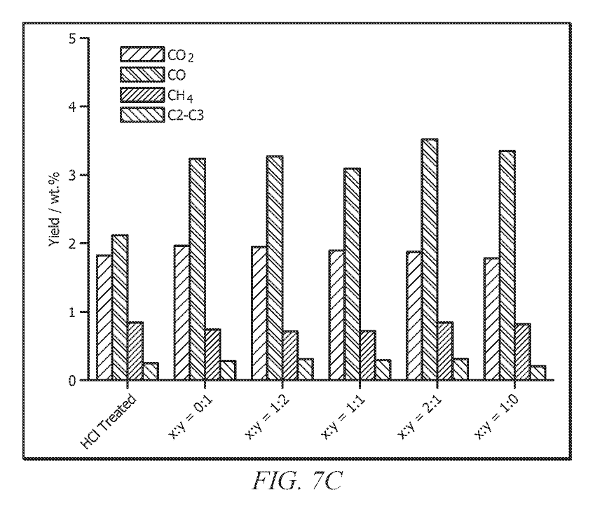

The conversion of lignin to a mixed phenol product using the methodologies disclosed herein was investigated. The conditions for pyrolysis of lignin were the indicated temperature for 20 seconds using a temperature ramp of 20° C./ms. The zeolite catalyst used was a combination of faujasite having a Si/Al ratio of 15 and zeolite Beta with a Si/Al ratio of 33 at a lignin:catalyst ratio of 1:1. The ratio of faujasite (designated x in the figures) to zeolite Beta (designated y in the figure) was 0:1, 1:2, 1:1, 2:1 or 1:0 and compared to an HCl-treated Kraft lignin reacted in the absence of catalyst. FIG. 7a provides for the yield of solid, liquid, and gas components at the indicated faujasite: zeolite Beta ratio while FIG. 7b displays the liquid product distribution and FIG. 7c provides the gas product distribution.

### Additional Disclosure

The following enumerated embodiments are provided as non-limiting examples.

A first embodiment which is a method for converting lignin to a phenol product, the method comprising contacting a zeolite catalyst with a lignin under reaction conditions sufficient to produce the phenol product at a yield of equal to or greater than about 50%.

A second embodiment which is the method of the first embodiment wherein the lignin comprises Kraft lignin, Organosolv lignin, lignosulfonate lignin, steam explosion lignin, or combinations thereof.

A third embodiment which is the method of any of the first through second embodiments wherein the lignin comprises Kraft lignin.

A fourth embodiment which is the method of any of the first through third embodiments wherein the lignin is pretreated with an acid prior to contact with the zeolite catalyst.

A fifth embodiment which is the method of the fourth embodiment wherein the acid comprises hydrochloric acid, phosphoric acid, sulfonic acid, or combinations thereof.

A sixth embodiment which is the method of the fourth embodiment wherein the acid pretreatment comprises contacting lignin with acid in an amount ranging from about 0.01 g acid/g lignin to about 0.22 g acid/g lignin at a temperature ranging from about 20° C. to about 70° C. for a time period of from about 2 hours to about 12 hours.

A seventh embodiment which is the method of any of the first through sixth embodiments wherein the zeolite catalyst has a pore opening diameter of from about 5 Å to about 10 Å.

An eighth embodiment which is the method of any of the first through seventh embodiments wherein the zeolite catalyst has a particle size of from about 0.05 μm to about 2 μm.

A ninth embodiment which is the method of any of the first through eighth embodiments wherein the zeolite catalyst comprises ZSM-5, ZSM-11, ZSM-23, Silicalite, Mordenite, zeolite Beta, Zeolite-L, Zeolite X and Y, faujasite, or combinations thereof.

A tenth embodiment which is the method of any of the first through ninth embodiments wherein the zeolite catalyst has a silia:alumina ratio of from about 5 to about 80.

An eleventh embodiment which is the method of any of the first through tenth embodiments wherein reaction conditions sufficient to produce the phenol product comprise a reaction temperature of from about 550° C. to about 850° C.

A twelfth embodiment which is the method of any of the first through eleventh embodiments further comprising recovering the phenol product.

A thirteenth embodiment which is the method of the twelfth embodiment wherein the recovered phenol product is further processed to produce a petrochemical.

A fourteenth embodiment which is the method of the twelfth embodiment wherein the recovered phenol product is further processed to produce a polymer.

A fifteenth embodiment which is a method for converting lignin to a mixed phenol product, the method comprising contacting a large-pore zeolite catalyst with a Kraft lignin under reaction conditions comprising a reaction temperature of from about 550° C. to about 850° C. to produce the mixed phenol product at a yield of equal to or greater than about 50%.

A sixteenth embodiment which is a method of the fifteenth embodiment wherein the large-pore zeolite catalyst has a pore opening diameter of from about 7 Å to about 10 Å.

A seventeenth embodiment which is a method of any of the fifteenth through sixteenth embodiments wherein the large-pore zeolite catalyst comprises faujasite, zeolite Beta, or combinations thereof.

An eighteenth embodiment which is the method of any of the fifteenth through seventeenth embodiments wherein the zeolite catalyst has a silia:alumina ratio of from about 5 to about 80.

A nineteenth embodiment which is the method of any of the fifteenth through eighteenth embodiments wherein the lignin is pretreated with hydrochloric acid prior to contact with the large pore zeolite catalyst.

A twentieth embodiment which is the method of any of the fifteenth through nineteenth embodiments further comprising recovering the mixed phenol product.

While embodiments of the present disclosure have been shown and described, modifications thereof can be made by one skilled in the art without departing from the spirit and teachings of the disclosure. The embodiments described herein are exemplary only, and are not intended to be limiting. Many variations and modifications of the disclosure are possible and are within the scope of the invention. Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The discussion of a reference in the Background is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed above and throughout the text are provided solely for their disclosure prior to the filing date of the present application. Nothing herein is to be construed as an admission that the inventors are not entitled to antedate such disclosure by virtue of prior invention.

Unless indicated otherwise, when a range of any type is disclosed or claimed it is intended to disclose or claim individually each possible number that such a range could reasonably encompass, including any sub-ranges encompassed therein. When describing a range of measurements every possible number that such a range could reasonably encompass can, for example, refer to values within the range with one significant digit more than is present in the end points of a range. Moreover, when a range of values is disclosed or claimed, which Applicants intent to reflect individually each possible number that such a range could reasonably encompass, Applicants also intend for the disclosure of a range to reflect, and be interchangeable with, disclosing any and all sub-ranges and combinations of sub-ranges encompassed therein. Accordingly, Applicants reserve the right to proviso out or exclude any individual members of any such group, including any sub-ranges or combinations of sub-ranges within the group, if for any reason Applicants choose to claim less than the full measure of the disclosure.

What is claimed is:

1. A method for converting lignin to a phenol product, the method comprising contacting a zeolite catalyst with a lignin under reaction conditions sufficient to produce the phenol product at a yield of equal to or greater than about 50%.

2. The method of claim 1 wherein the lignin comprises Kraft lignin, Organosolv lignin, lignosulfonate lignin, steam explosion lignin, or combinations thereof.

3. The method of claim 1 wherein the lignin comprises Kraft lignin.

4. The method of claim 1 wherein the lignin is pretreated with an acid prior to contact with the zeolite catalyst.

5. The method of claim 4 wherein the acid comprises hydrochloric acid, phosphoric acid, sulfonic acid, or combinations thereof.

6. The method of claim 4 wherein the acid pretreatment comprises contacting lignin with acid in an amount ranging from about 0.01 g acid/g lignin to about 0.22 g acid/g lignin at a temperature ranging from about 20° C. to about 70° C. for a time period of from about 2 hours to about 12 hours.

7. The method of claim 1 wherein the zeolite catalyst has a pore opening diameter of from about 5 Å to about 10 Å.

8. The method of claim 1 wherein the zeolite catalyst has a particle size of from about 0.05 μm to about 2 μm.

9. The method of claim 1 wherein the zeolite catalyst comprises ZSM-5, ZSM-11, ZSM-23, Silicalite, Mordenite, zeolite Beta, Zeolite-L, Zeolite X and Y, faujasite, or combinations thereof.

10. The method of claim 1 wherein the zeolite catalyst has a silia:alumina ratio of from about 5 to about 80.

11. The method of claim 1 wherein reaction conditions sufficient to produce the phenol product comprise a reaction temperature of from about 550° C. to about 850° C.

12. The method of claim 1 further comprising recovering the phenol product.

13. The method of claim 12 wherein the recovered phenol product is further processed to produce a petrochemical.

14. The method of claim 12 wherein the recovered phenol product is further processed to produce a polymer.

15. A method for converting lignin to a mixed phenol product, the method comprising contacting a large-pore zeolite catalyst with a Kraft lignin under reaction conditions comprising a reaction temperature of from about 550° C. to about 850° C. to produce the mixed phenol product at a yield of equal to or greater than about 50%.

16. The method of claim 15 wherein the large-pore zeolite catalyst has a pore opening diameter of from about 7 Å to about 10 Å.

17. The method of claim 15 wherein the large-pore zeolite catalyst comprises faujasite, zeolite Beta, or combinations thereof.

18. The method of claim 15 wherein the zeolite catalyst has a silia:alumina ratio of from about 5 to about 80.

19. The method of claim 15 wherein the lignin is pretreated with hydrochloric acid prior to contact with the large pore zeolite catalyst.

20. The method of claim 15 further comprising recovering the mixed phenol product.
